(19) United States
(12) Patent Application Publication
(10) Pub. No.: US 2016/0242953 A1
(43) Pub. Date: Aug. 25, 2016

(54) VAGINAL LAXITY AND UTERINE PROLAPSE DEVICE

(71) Applicant: Charles R. Bridges, Charlotte, NC (US)

(72) Inventor: Charles R. Bridges, Charlotte, NC (US)

(21) Appl. No.: 14/630,904

(22) Filed: Feb. 25, 2015

(51) Int. Cl. A61F 6/12

(52) U.S. Cl. CPC A61F 6/12

(57) ABSTRACT

A vaginal laxity/uterine prolapse system is disclosed. The system includes vaginal laxity/uterine prolapse device and a suction device. The vaginal laxity/uterine prolapse device includes a shaft sized and configured for insertion into a patient's vagina. The shaft includes a plurality of suction ports. The shaft is coupled to a suction device to generate suction through the plurality of suction ports. The suction causes edema and increased blood flow to the vagina walls, resulting in an immediate increase in vaginal tone (“tightness”) and over time, with repeated use, causes hypertrophy of the vaginal wall and associated pelvic musculature, resulting in a permanent increase in vaginal tightness, significantly increasing the friction resistance to penile penetration. Use of the vaginal laxity/uterine prolapse system results in immediate and long-term increase in the frictional resistance and improved pelvic and vaginal muscle strength to prevent retrograde migration of the uterus into the vagina (uterine prolapse).

### BACKGROUND

Some medical procedures result in looseness/loss of tightness in the vaginal and/or pelvic muscles. For example, after child birth, many women experience looseness in the vaginal and/or pelvic floor muscles that can result in uncontrolled urine discharge (for example as a result of sneezing), loss of feeling, and/or loss of sexual fulfillment. In addition, many women experience anxiety regarding looseness after child birth. Many exercises have been developed for tightening of the pelvic floor and/or vaginal muscles, such as, for example, Kegel exercises. These exercises require prolonged and frequent use in order to produce noticeable results.

### SUMMARY

In various embodiments, a vaginal laxity/uterine prolapse system is disclosed. The vaginal laxity/uterine prolapse system comprises a vaginal laxity/uterine prolapse device and a suction device. The vaginal laxity/uterine prolapse device comprises a shaft extending from a proximal end to a distal end along a longitudinal axis. The shaft is sized and configured for insertion into a body cavity. The proximal end of the shaft is sized and configured to seal the body cavity when the shaft is inserted. The shaft defines an internal channel. A plurality of suction holes are formed through the shaft. The plurality of suction holes extend through a surface of the shaft to allow fluid communication between an outer surface of the shaft and the internal channel. The suction device is coupled to the internal channel and is configured to generate suction through the plurality of holes in the shaft.

In various embodiments, a vaginal laxity/uterine prolapse device is disclosed. The vaginal laxity/uterine prolapse device comprises a shaft extending from a proximal end to a distal end along a longitudinal axis. The shaft is sized and configured for insertion into a body cavity. The proximal end of the shaft is sized and configured to seal the body cavity when the shaft is inserted. The shaft defines an internal channel. A plurality of suction holes are formed through the shaft. The plurality of suction holes extend through a surface of the shaft to allow fluid communication between an outer surface of the shaft and the internal channel.

In various embodiments, a method for generating an edema and hypertrophy in a vaginal wall is disclosed. The method comprises a first step of inserting a vaginal laxity/uterine prolapse device into a body cavity. The vaginal laxity/uterine prolapse device comprises a shaft extending from a proximal end to a distal end, a handle coupled to a proximal end of the shaft, and a sealing cup located between the handle and the shaft. The shaft defines an internal channel. A plurality of suction holes are formed through the shaft. The sealing cup is configured to seal a cavity opening when the shaft is inserted into the body cavity. The method further comprises applying suction to a wall of the body cavity. Suction is applied by the prolapse device through the plurality of holes formed on the shaft.

### BRIEF DESCRIPTION OF THE FIGURES

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates one embodiment of a vaginal laxity/uterine prolapse system.

FIG. 2 illustrates one embodiment of a vaginal laxity/uterine prolapse system including a vaginal laxity/uterine prolapse device having a suction device in the handle.

FIG. 3 illustrates one embodiment of a vaginal laxity/uterine prolapse device having a self-contained suction device located within the handle.

FIG. 4 illustrates one embodiment of a vaginal laxity/uterine prolapse device including a self-contained suction device and an interior protective cup.

FIG. 5 illustrates one embodiment of a vaginal laxity/uterine prolapse system including a vaginal laxity/uterine prolapse device having a handle defining an entrance seating profile.

FIG. 6 illustrates one embodiment of a vaginal laxity/uterine prolapse system including a vaginal laxity/uterine prolapse device having a handle defining an entrance seating profile and a clitoral stimulator.

FIG. 7 illustrates one embodiment of a vaginal laxity/uterine prolapse system including a vaginal laxity/uterine prolapse device having an one or more electrodes formed on the shaft.

FIG. 8 illustrates one embodiment of a vaginal laxity/uterine prolapse system including a vaginal laxity/uterine prolapse device having an interior protective cup, one or more electrodes, and suction device located in the handle housing.

FIG. 9A illustrates one embodiment of a vaginal laxity/uterine prolapse device including a shaft having one or more suction ports.

FIG. 9B illustrates one embodiment of a suction distribution cap configured to interface with the shaft of the vaginal laxity/uterine prolapse device of FIG. 9A.

FIG. 9C illustrates a side view of the suction distribution cap of FIG. 9B.

FIG. 9D illustrates one embodiment of a hand pump configured to couple to the vaginal laxity/uterine prolapse device of FIG. 9A.

FIG. 9E illustrates one embodiment of a vaginal laxity/uterine prolapse system including the vaginal laxity/uterine prolapse device of FIG. 9A and the hand pump of FIG. 9D.

FIG. 10A illustrates one embodiment of an electric pump sphere configured to generate suction in a vaginal laxity/uterine prolapse device.

FIG. 10B illustrates one embodiment of a vaginal laxity/uterine prolapse device configured to couple to the electric pump sphere of FIG. 10A.

FIG. 11A illustrates one embodiment of a vaginal laxity/uterine prolapse device including a protective blossom cap.

FIG. 11B illustrates one embodiment of the vaginal laxity/uterine prolapse device of FIG. 11A with a protective blossom in a deployed position.

FIG. 12A illustrates one embodiment of a vaginal laxity/uterine prolapse device including a protective blossom spreading stem.

FIG. 12B illustrates one embodiment of the vaginal laxity/uterine prolapse device of FIG. 12A having a protective blossom wrapped around the spreading stem.

FIG. 12C illustrates one embodiment of the vaginal laxity/uterine prolapse device of FIG. 12A having the protective blossom in a deployed position.

FIGS. 13A-13B illustrate one embodiment of a vaginal laxity/uterine prolapse device coupled to a manual hand pump.

FIGS. 13C-13E illustrate one embodiment of the manual hand pump of FIG. 13A in various pumping stages.

FIG. 14A illustrates one embodiment of a vaginal laxity/uterine prolapse system including a vaginal laxity/uterine prolapse device coupled to a hand pump.

FIGS. 14B-14C illustrate the hand pump of FIG. 14A in various pumping stages.

FIG. 14D illustrates the vaginal laxity/uterine prolapse device of FIG. 14A receiving suction.

FIG. 15 illustrates one embodiment of a vaginal laxity/uterine prolapse system including a vaginal laxity/uterine prolapse device coupled to a wirelessly controlled electric pump.

FIGS. 16A-16D illustrate embodiments of vaginal laxity/uterine prolapse devices having various suction port patterns.

FIG. 17A illustrates one embodiment of a disposable suction distribution cover configured to couple to the shaft of a vaginal laxity/uterine prolapse device.

FIG. 17B illustrates one embodiment of a vaginal laxity/uterine prolapse device having the disposable cover of FIG. 17A coupled thereto.

FIGS. 18A-18C illustrate one embodiment of a quick disconnect system for coupling a vaginal laxity/uterine prolapse device to a suction device.

FIG. 19 illustrates one embodiment of a pressure measuring device configured to measure progress of a prolapse generated by a vaginal laxity/uterine prolapse device.

FIG. 20 is a flowchart illustrating one embodiment of a method for generating edema and/or hypertrophy of a vaginal wall.

### DETAILED DESCRIPTION

The description of the preferred embodiments is intended to be read in connection with the accompanying drawings, which are to be considered part of the entire written description of this invention. The drawing figures are not necessarily to scale and certain features of the invention may be shown exaggerated in scale or in somewhat schematic form in the interest of clarity and conciseness. In this description, relative terms such as “horizontal,” “vertical,” “up,” “down,” “top,” “bottom,” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral,” “proximal” versus “distal” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both moveable or rigid attachments or relationships, unless expressly described otherwise. The term “operatively coupled” is such an attachment, coupling, or connection that allows the pertinent structures to operate as intended by virtue of that relationship. In the claims, means-plus-function clauses, if used, are intended to cover structures described, suggested, or rendered obvious by the written description or drawings for performing the recited function, including not only structure equivalents but also equivalent structures.

In various embodiments, a vaginal laxity/uterine prolapse system is disclosed. The vaginal laxity/uterine prolapse system generally comprises a vaginal laxity/uterine prolapse device and a suction device. The vaginal laxity/uterine prolapse device generally includes a shaft sized and configured for insertion into a patient's vagina. The shaft includes a plurality of suction ports. The shaft is coupled to a suction device configured to generate suction in the vagina through the plurality of suction ports. The suction within the vagina causes both immediate and chronic decrease in the compliance of the vagina, increasing the frictional resistance to penile penetration (e.g., “tightness”) to treat vaginal laxity and conversely increasing the frictional resistance to retrograde displacement of the uterus to decrease the frequency and severity of vaginal prolapse. In some embodiments, repeated use of the vaginal laxity/uterine prolapse device results in permanent tightening of the vagina and/or a vaginal opening.

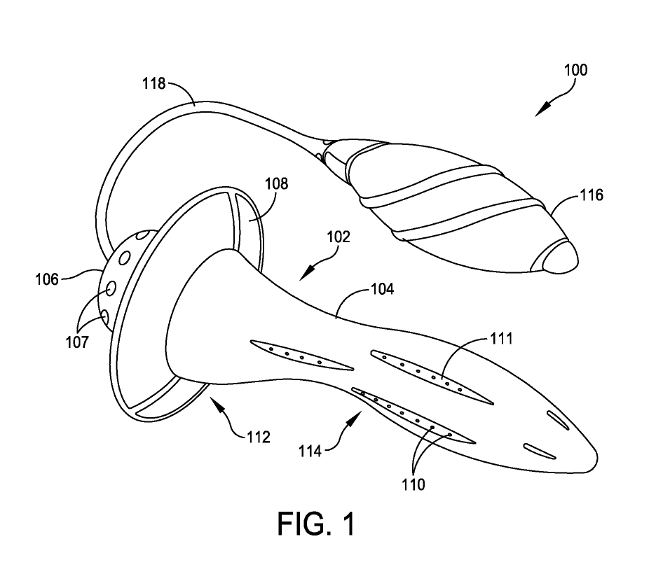

FIG. 1 illustrates one embodiment of a vaginal laxity/uterine prolapse system 100. The vaginal laxity/uterine prolapse system 100 includes a vaginal laxity/uterine prolapse device 102. The vaginal laxity/uterine prolapse device 102 comprises a shaft 104 extending longitudinally along a shaft axis. The shaft 104 is sized and configured for insertion into a body cavity of a patient, such as, for example, a vagina. In some embodiments, the shaft 104 comprises a rigid shaft. The shaft 104 is configured to extend from a vaginal opening a predetermined distance into the vagina. In some embodiments, the shaft 104 has a length approximately equal to the average distance from the labial folds to the cervix, such as, for example, about 7 inches. In some embodiments, the length of the shaft 104 is adjustable.

In some embodiments, the shaft 104 comprises a profile sized and configured to partially or completely seal the vaginal opening after insertion. For example, the shaft 104 comprises a proximal section 112 having a diameter greater than the diameter of the remainder of the shaft 104. The increased diameter of the proximal section 112 is larger than the unexpanded diameter of the vaginal opening when the shaft 104 is inserted into the user's vagina. The proximal section 112 contacts the wall of the vaginal opening and seals the vaginal opening. In some embodiments, the shaft 104 comprises a distal section 114 configured to allow easy insertion of the vaginal laxity/uterine prolapse device 102 into the vaginal opening. For example, in some embodiments, the distal section 114 of the shaft 104 has a maximum diameter that is substantially less than an average penile diameter, such as, for example, a ¼″.

The shaft 104 is coupled to a handle (or base) section 106. The handle 106 is sized and configured to allow a user to grip the vaginal laxity/uterine prolapse device 102 during insertion/removal. The handle 106 is configured to remain outside of a user. In some embodiments, the handle 106 comprises one or more vent slots 107 configured to allow air flow into/out of the shaft 104 (described in more detail below.) In some embodiments, a sealing cup (or suction cup) 108 is located between the shaft 104 and the handle 106. The sealing cup 108 is located proximally of the proximal section 112 of the shaft 104. The sealing cup 108 is sized and configured to contact and seal the vaginal opening. In some embodiments, the sealing cup 108 is sized and configured to fit completely over the vaginal opening, the labial lips, and/or the pelvic area of a user. The sealing cup 108 fits over at least the vaginal opening and seals the outer surface of the vaginal opening. In some embodiments, the sealing cup 108 is positioned such that the sealing cup 108 covers the vaginal opening when the proximal section 112 of the shaft 104 is in contact with the walls of the vaginal opening. In some embodiments, the sealing cup 108 may be similar to a contraceptive diaphragm. The sealing cup 108 can comprise a concave, convex, and/or flat contact profile and may comprise any suitable material, such as, for example, plastic and/or rubber.

In some embodiments, the shaft 104 includes a plurality of suction openings 110 distributed over the length of the shaft 104. The plurality of suction openings 110 each include one or more channels or holes formed through the shaft 104. The shaft 104 defines an inner channel (not shown) in fluid communication with the one or more channels of the suction openings 110. In some embodiments, the plurality of suction openings 110 are located within one or more suction tracts 111. The suction openings 110 allow air flow between the vagina and a volume of air outside of the patient. Air flow from within the vagina to the outer volume generates a vacuum within the vagina, causing increased vaginal blood flow and/or muscle contractions. The increased vaginal blood flow generates an edema and/or hypertrophy of the vaginal walls.

The plurality of suction openings 110 may be disposed in any suitable pattern, such as, for example, a symmetrical pattern, a non-symmetrical pattern, a random pattern, and/or any other suitable pattern. The plurality of suction openings 110 may be located on only a portion of the shaft 104 and/or over the entire length of the shaft. For example, in various embodiments, the plurality of suction openings are located only on the distal section 114, only on the proximal section 112, and/or on any other suitable section of the shaft 104. In some embodiments, the plurality of suction openings 110 comprise different diameters. The diameter of a specific suction opening 110 may depend on, for example, the location of the suction opening 110 on the shaft 104. For example, in some embodiments, the diameters of the suction openings 110 may change uniformly over the length of the shaft 104.

The plurality of suction openings 110 are in fluid communication with an internal channel defined by the shaft 104. For example, in some embodiments, the shaft 104 is hollow and the entire inner volume comprises an internal channel. In other embodiments, the shaft 104 is semi-solid and has an internal channel formed therein. The internal channel is coupled to the handle section 106. In some embodiments, suction is generated by a suction device 116. The suction device may be located within the handle section 106 and/or coupled to the prolapse device 102 by a suction tube 108 (as shown in FIG. 1). In the illustrated embodiment, a hand pump 116 is coupled to the handle section 106 by a suction tube 118. Actuation of the hand pump 116 causes air to flow from the vagina, through the plurality of suction openings 110 and the inner channel, and out of the one or more vent slots 107 formed in the handle 106. The flow of air out of the vaginal laxity/uterine prolapse device 102 generates suction within the vagina to draw the vaginal walls towards the shaft 104 of the vaginal laxity/uterine prolapse device 102.

The negative pressure within the vagina increases vaginal blood flow. The increased blood flow causes edema of the vaginal walls and/or hypertrophy of the vaginal wall muscle. In some embodiments, repeated and/or prolonged use, such as, for example, as part of a treatment regimen, inhibits vaginal laxity/uterine prolapse, causes permanent hypertrophy of the vaginal walls/muscles, and/or increases sexual pleasure of the patient as a result of the increased blood flow, muscle thickness, and reduced size of the vagina. In some embodiments, the vaginal laxity/uterine prolapse system 100 comprises a novelty/periodic use device configured to temporarily increase blood flow, vaginal tightness, and sensitivity to increase sexual enjoyment for a user and/or a partner. In other embodiments, the vaginal laxity/uterine prolapse system 100 comprises a medical device configured for medical treatment of vaginal laxity/uterine prolapse and/or to permanently increase vaginal tightness and/or hypertrophy, for example, after child birth.

In some embodiments, the vaginal laxity/uterine prolapse system 100 is used in conjunction with one or more rehabilitative exercises. For example, in some embodiments, the vaginal laxity/uterine prolapse system 100 is combined with pelvic floor exercises, such as, for example kegel exercises, to increase vaginal tightness and/or blood flow to the vaginal area. In some embodiments, the vaginal laxity/uterine prolapse system 100 is configured to be used simultaneously with the rehabilitative exercises, e.g., kegel exercises may be performed during application of suction by the vaginal laxity/uterine prolapse device 102. In other embodiments, the vaginal laxity/uterine prolapse system 100 is used in conjunction with the rehabilitative exercises, e.g., the vaginal laxity/uterine prolapse device may be used between kegel exercise sessions.

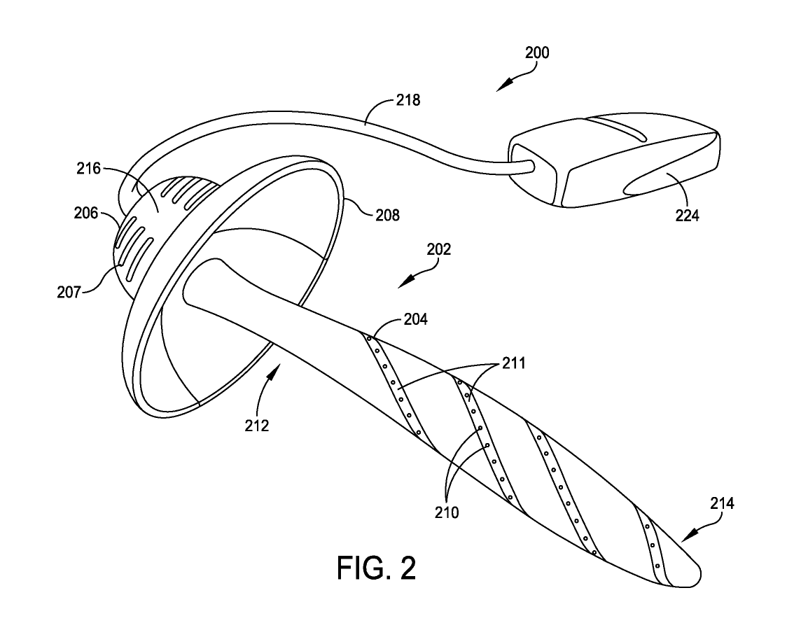

FIG. 2 illustrates one embodiment of a vaginal laxity/uterine prolapse system 200 including a vaginal laxity/uterine prolapse device 202 having a suction device 216 in the handle 206. The vaginal laxity/uterine prolapse device 202 is similar to the vaginal laxity/uterine prolapse device 102 discussed above with respect to FIG. 1. The vaginal laxity/uterine prolapse device 202 comprises a shaft 204 having a spiral suction pattern 211. The spiral suction pattern 211 comprises a continuous groove formed in the shaft 204 extending from the distal end 214 to the proximal end 212. A plurality of suction holes 210 are contained within the spiral suction pattern 211 and allow fluid communication between the outer surface of the shaft 204 and an inner channel (not shown) that extends from a distal end 214 of the shaft 204 to the handle 206.

In some embodiments, the handle 206 contains a suction device 216 that is coupled to the inner channel of the shaft 204. The suction device is configured to generate air flow through the plurality of suction holes 210, the inner channel, and out of the plurality of vent holes 207 formed on the handle 206. The suction device 216 is configured to generate suction within the vagina when the shaft 204 is inserted therein. In some embodiments, the suction device 216 is reversible, for example, to generate air flow into the vagina to release the vaginal wall from the shaft 204. In some embodiments, the suction device 216 comprises an electric fan. A sealing cup 208 is disposed between the shaft 204 and the handle 206.

In some embodiments, the suction device 216 is controlled by a controller 224. The controller 224 may be coupled to the handle 206 by a cord 218. In some embodiments, the controller 224 is wireless. The controller 224 may be configured to start/stop the suction device, increase/decrease the speed/suction of the suction device, and/or to reverse the direction of air flow. In some embodiments, the controller 224 includes a display, such as, for example, a digital display, to display information to a user, such as, for example, a timer, a suction measurement, and/or any other suitable information.

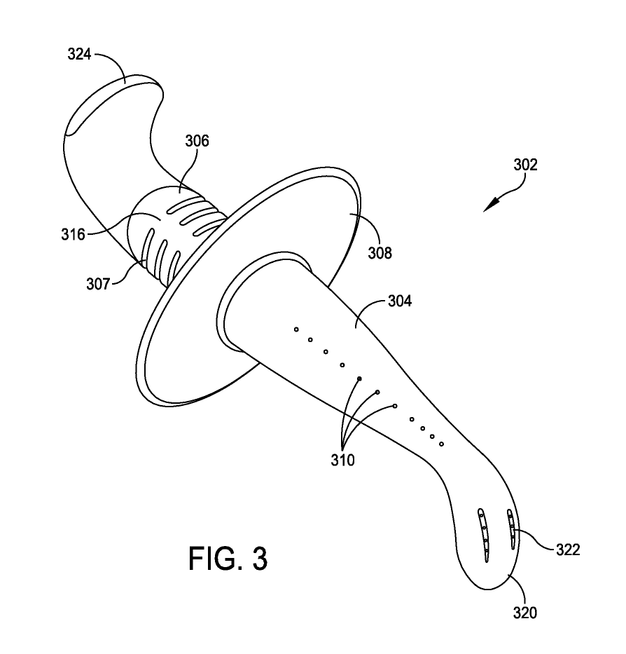

FIG. 3 illustrates one embodiment of a vaginal laxity/uterine prolapse device 302 having a self-contained suction assembly 316 coupled to the handle 306. The vaginal laxity/uterine prolapse device 302 is similar to the vaginal laxity/uterine prolapse devices described above. The vaginal laxity/uterine prolapse device 302 includes a shaft 304. A plurality of suction openings 310 are formed along the length of the shaft 304. The plurality of suction openings 310 are configured to allow fluid communication between the outer surface of the shaft 304 and an inner channel defined by the shaft 304. The inner channel is coupled to the handle 306.

A distal head section 320 is coupled to the shaft 304 at an angle. For example, in the illustrated embodiment, the distal head section 320 is coupled to the shaft 304 at an angle of about 90°. The distal head section 320 comprises a suction pattern 322 formed thereon. The suction pattern 322 may comprise any suitable pattern, such as, for example, elongate channels formed on the distal head section 320. The suction pattern 322 includes a plurality of channels 310 in fluid communication with the internal channel defined by the shaft 304.

In some embodiments, the vaginal laxity/uterine prolapse device 302 comprises a handle 306. The handle 306 includes a suction device 316 located therein. The suction device is configured to generate air flow through the plurality of openings 310, the inner channel, and out of the plurality of vent holes 307 formed on the handle 306. The suction device 316 is configured to generate suction within the vagina when the shaft 304 is inserted therein. In some embodiments, the suction device 316 is reversible, for example, to generate air flow into the vagina to release the vaginal wall from the shaft 304. A sealing cup 308 is disposed between the shaft 304 and the handle 306.

In some embodiments, a controller 324 is coupled to the handle 306. The controller 324 includes one or more controls for the suction device. For example, in various embodiments, the controller 324 may be configured to control a start/stop of the suction device, the speed/suction of the suction device, and/or the direction of air flow. The controller 324 may be configured to control the suction device through any suitable control scheme, such as, for example, one or more buttons located on the controller 324, rotating the controller 324 with respect to the handle 306, and/or any other suitable control scheme. The controller 324 controls the suction generated within the vagina by controlling air flow out of the vagina. In some embodiments, the controller 324 includes a housing sized and configured to extend the handle 306.

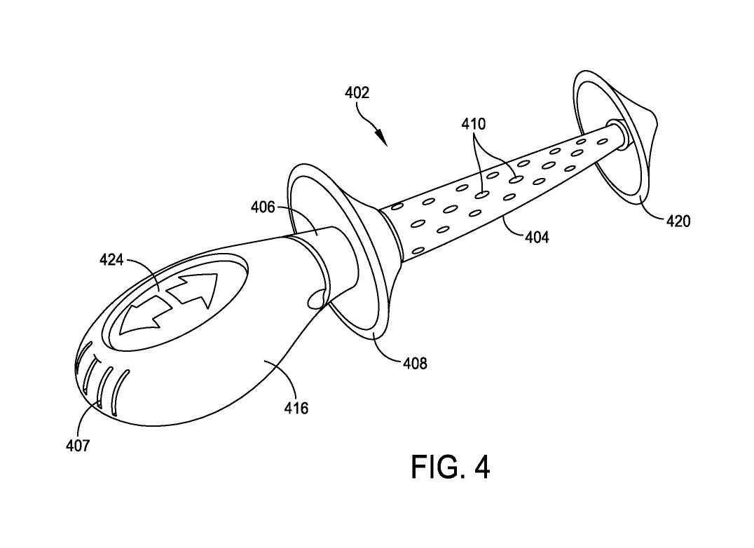

FIG. 4 illustrates one embodiment of a vaginal laxity/uterine prolapse device 402 including a self-contained suction assembly 416 and an interior protective cup 424. The vaginal laxity/uterine prolapse device 402 includes a shaft 404 extending longitudinally along a shaft axis. The shaft 404 is sized and configured for insertion into a vaginal opening. The shaft 404 extends a predetermined length corresponding to, for example, the average length of a vagina. In some embodiments, the length of the shaft 404 is adjustable. A convex sealing cup 408 is located at the proximal end of the shaft 404. The sealing cup 408 is sized and configured to seal the vaginal opening after insertion of the shaft 404. The sealing cup 408 may comprise a plastic and/or rubber cup sized and configured to fit partially within and/or overtop of the vaginal opening and/or the pelvic region of a patient.

In some embodiments, the shaft 404 comprises a plurality of suction channels 410. The plurality of suction channels 410 are disposed along the length of the shaft 404 and allow fluid communication between the outer surface of the shaft 404 and an internal channel defined by the shaft 404. The plurality of suction channels 410 may comprise uniform suction channels and/or non-uniform suction channels. The plurality of suction channels 410 are configured to generate suction within the vagina. The plurality of suction channels 410 allow fluid (such as, for example, air) to flow from within the vagina, through the inner channel, and out of the plurality of vents 407 formed on the suction device 416.

In some embodiments, an interior protective cup 424 is located at a distal end of the shaft 404. The interior protective cup 424 is sized and configured to protect the internal anatomy of a patient. For example, in some embodiments, the interior protective cup 424 is a uterine protective cup sized and configured to fit over the cervix within a vagina. The interior protective cup 424 protects the internal anatomy, such as the uterus and cervix, of the patient from the negative pressure generated by the plurality of suction channels 410. In some embodiments, the interior protective cup 424 comprises a soft, cuplike structure made of a material, such as, for example rubber, configured to contact the distal wall of the vagina. The interior protective cup 424 may be deformable to assist in insertion of the vaginal laxity/uterine prolapse device 402. The interior protective cup 424 resumes an un-deformed shape after insertion. In some embodiments, the interior protective cup 424 is similar to a contraceptive diaphragm.

In some embodiments, the vaginal laxity/uterine prolapse device 402 comprises a self-contained suction device 416 coupled to the distal end of the shaft 404. The self-contained suction device 416 is configured to generate negative pressure through the plurality of suction channels 410. The self-contained suction device 416 may comprise, for example, a fan-powered suction device. In some embodiments, the self-contained suction device includes a housing sized and configured as a handle of the vaginal laxity/uterine prolapse device 402. The self-contained suction device 416 includes a plurality of vent channels 407. The plurality of vent channels 407 are in fluid communication with the inner channel defined by the shaft 404. When the self-contained suction device 416 is activated, air flows from the internal vagina, through the plurality of suction channels 410 and the inner channel defined by the shaft 404. The air passes through the self-contained suction device 416 and is vented through the plurality of vent slots 406.

In some embodiments, the self-contained suction device 416 comprises a controller 424. The controller 424 controls one or more features of the self-contained suction device 416, such as, for example, turning the suction device 416 on/off, adjusting the suction level of the suction device 416, and/or changing the direction of air flow of the suction device 416. In some embodiments, the controller 424 comprises a rocker switch configured to increase/decrease the suction level of the suction device 416 from a minimum (or off-state) to a maximum suction level.

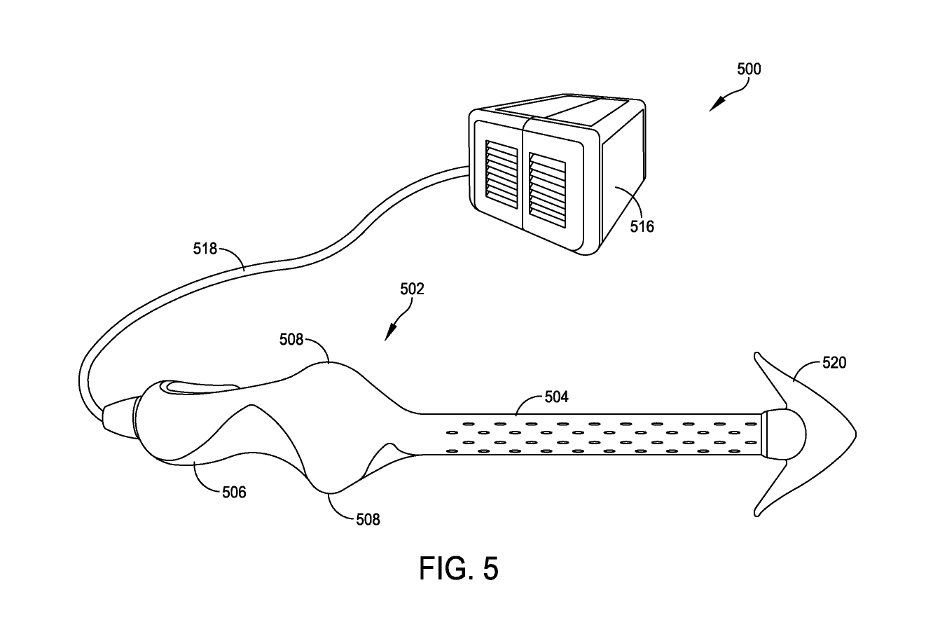

FIG. 5 illustrates one embodiment of a vaginal laxity/uterine prolapse system 500 including a vaginal laxity/uterine prolapse device 502 having a handle 506 defining an entrance seating profile 508. The vaginal laxity/uterine prolapse device 502 is similar to the vaginal laxity/uterine prolapse device discussed above with respect to FIGS. 1-4. The vaginal laxity/uterine prolapse device 502 includes a shaft 504 extending along a longitudinal axis from a proximal end to a distal end. The proximal end of the shaft 504 includes an entrance seating profile 508. The entrance seating profile 508 is sized and configured to position the vaginal laxity/uterine prolapse device 502 and seal the vagina. The entrance seating profile 508 prevents fluid communication between the vagina of the patient and an outer volume. The entrance seating profile 508 seals the vagina to allow a negative pressure to be generated therein. In some embodiments, an internal protective cup 524 is disposed at a distal end of the shaft 504.

A suction device 516 is coupled to the handle 506 through a suction tube 518. The suction device 516 is configured to generate negative pressure through a plurality of suction channels 510 formed on the shaft 504. The suction device 516 may comprise, for example, a fan-powered pump. When the shaft 504 is inserted into the vagina, the suction device 516 is activated to generate negative pressure within the vagina and cause increased blood flow to the vaginal wall.

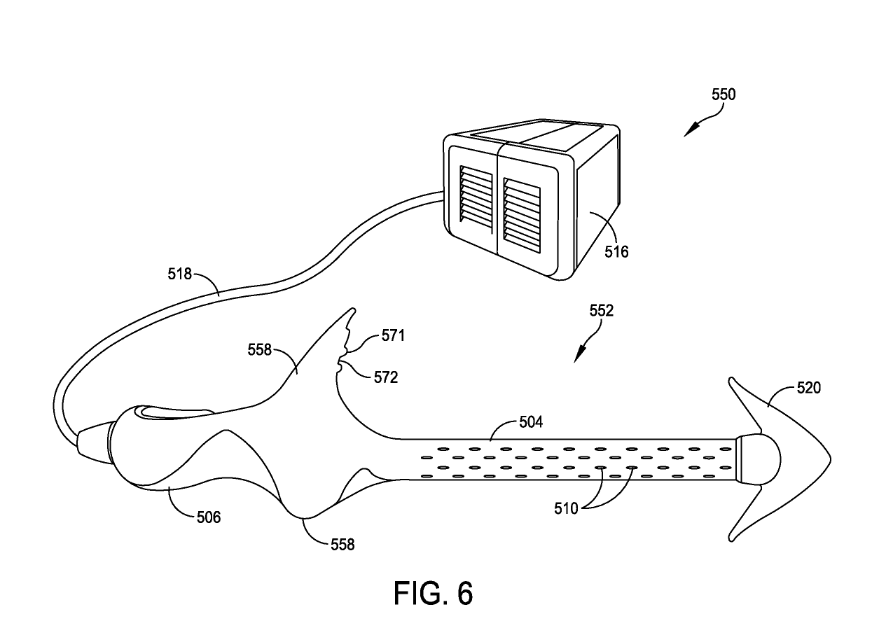

FIG. 6 illustrates one embodiment of a vaginal laxity/uterine prolapse system 550 comprising a vaginal laxity/uterine prolapse device 552 having a clitoral stimulator 572. The vaginal laxity/uterine prolapse system 550 is similar to the vaginal laxity/uterine prolapse system 500 described above with respect to FIG. 5. The vaginal laxity/uterine prolapse device 552 includes an entrance seating profile 558. The entrance seating profile 558 includes an external clitoral stimulator 572. The external clitoral stimulator 572 is configured to contact a clitoris when the shaft 504 is inserted into the vagina of a user. The external clitoral stimulator 572 remains outside of the vaginal opening. The entrance seating profile 558 and the external clitoral stimulator 572 seal the vaginal opening when the shaft 504 is inserted into the user's vagina. In some embodiments, when the suction device 516 is activated to generate negative pressure, the external clitoral stimulator vibrates due to air flow through the internal cavity. The external clitoral stimulator 572 stimulates the clitoris to increase vaginal blood flow of the user. In some embodiments, the external clitoral stimulator 572 includes one or more projections 571 configured to ensure contact between the clitoris and the external clitoral stimulator 572 and/or to increase stimulation of the clitoris.

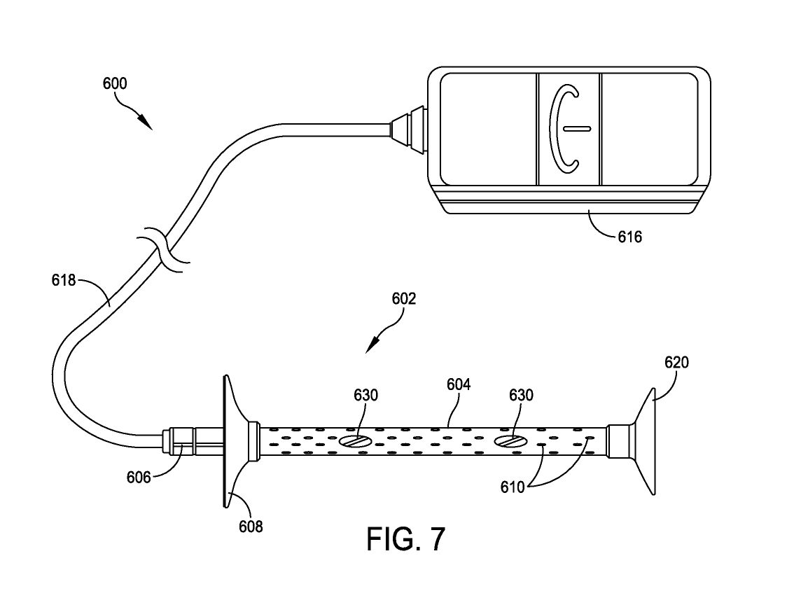

FIG. 7 illustrates one embodiment of a vaginal laxity/uterine prolapse system 600 including a vaginal laxity/uterine prolapse device 602 having an internal protective cup 620 and one or more electro-stimulation pads 630. The vaginal laxity/uterine prolapse device 602 includes a shaft 604 extending from a proximal end to a distal end along a longitudinal axis. The shaft 604 includes a plurality of suction channels 610 and defines an internal channel (not shown) in fluid communication with the plurality of suction channels 610. A handle 606 is coupled to a proximal end of the shaft 604. A sealing cup 608 is located between the handle 606 and the shaft 604. The vaginal laxity/uterine prolapse system 600 is similar to the vaginal laxity/uterine prolapse devices discussed above.

The vaginal laxity/uterine prolapse device 602 includes one or more electrodes 630 formed on the shaft 604. The electrodes 630 comprise electrical contacts configured to generate an electrical current in the vaginal wall of a patient to cause electro-stimulation of the vaginal wall and/or muscles located close to the vaginal wall. The electrodes 630 comprise, for example, contact electrodes formed on the shaft 604. In some embodiments, the electrodes 630 are coupled to a power source, for example, a suction/power controller 624, by one or more wires within the cable 618. In some embodiments, the electrodes 630 are configured to generate a bipolar current, e.g., a current that travels from the power source, through a first of the electrodes 630, through at least a portion of the vaginal wall, and returns to the power source through the second electrode 630. In some embodiments, the electrodes 630 are configured to generate a monopolar current in conjunction with a ground pad coupled to a patient. The monopolar current is generated at each of the electrodes 630 and travels through at least a portion of the vaginal wall and returns to the power source through a ground pad coupled externally to the patient.

In some embodiments, the electrodes 630 provide a current configured to constrict and/or relax one or more vaginal muscles. For example, in some embodiments, the electrodes 630 are configured to generate a bipolar current in at least a portion of the vaginal wall. The current generated in the vaginal wall causes one or more vaginal muscles to contract and relax repeatedly. The stimulation of the vaginal muscles over time causes tightening and/or increased muscle size. In some embodiments, the electrical current is generated simultaneously with the application of suction to the vaginal walls. In other embodiments, the electrical current may alternate with the suction.

A controller 624 is coupled to the vaginal laxity/uterine prolapse device 602 by a cord 618. In some embodiments, the cord 618 comprise a power cord and/or a suction tube to allow air flow. The controller 624 may comprise an electrical controller for controlling application of an electrical current through the electrodes 630 and/or a suction controller for controlling suction applied to the vaginal walls by the plurality of suction openings 610. In some embodiments, the controller 624 is configured to allow adjustments of the intensity of the current and/or the negative pressure generated by the vaginal laxity/uterine prolapse device 602.

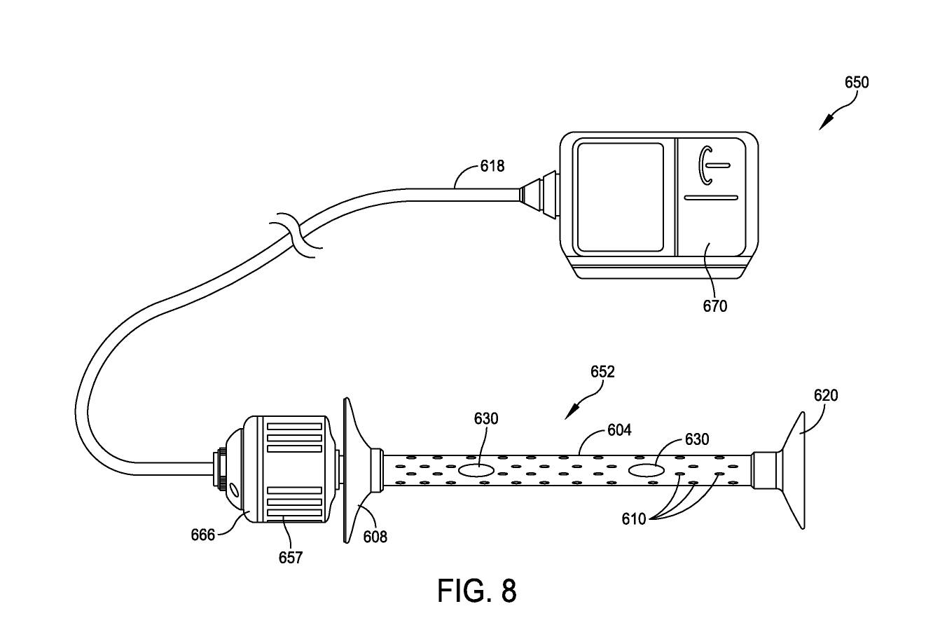

FIG. 8 illustrates one embodiment of a vaginal laxity/uterine prolapse system 650 including a vaginal laxity/uterine prolapse device 652 having an internal protection cup 620, one or more electrodes 630, and a self-contained suction device 666 configured as a handle. The vaginal laxity/uterine prolapse system 650 is similar to the vaginal laxity/uterine prolapse system 600 discussed above with respect to FIG. 7. The vaginal laxity/uterine prolapse system 650 comprises a vaginal laxity/uterine prolapse device 652 having a self-contained suction device 666 formed integrally therewith. The self-contained suction device 666 may comprise, for example, a fan having a housing 657. The self-contained suction device 666 is coupled to a controller 670 by a control cable 618. The controller 670 is configured to control suction generated by the self-contained suction device 666 and/or electrical current generated by and/or applied to the one or more electrodes 630.

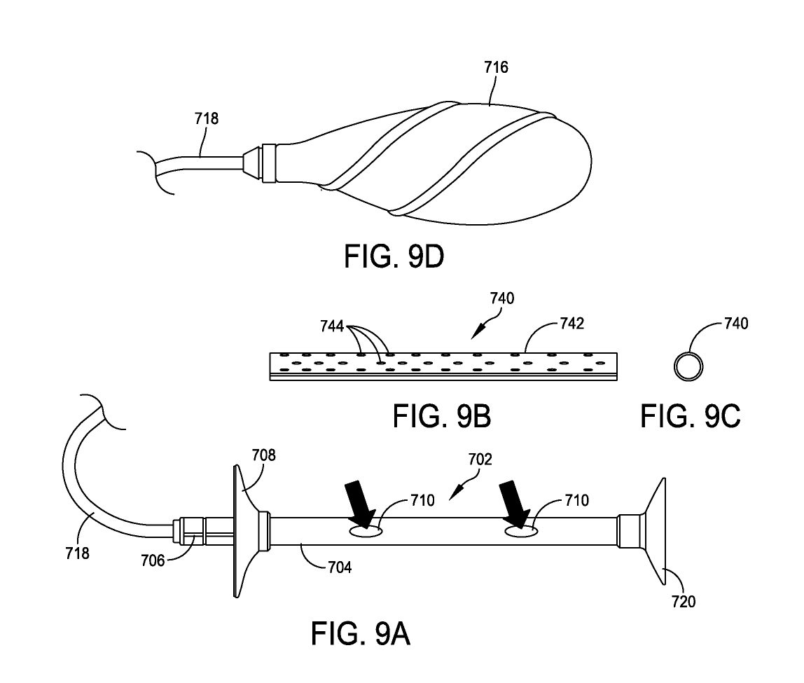

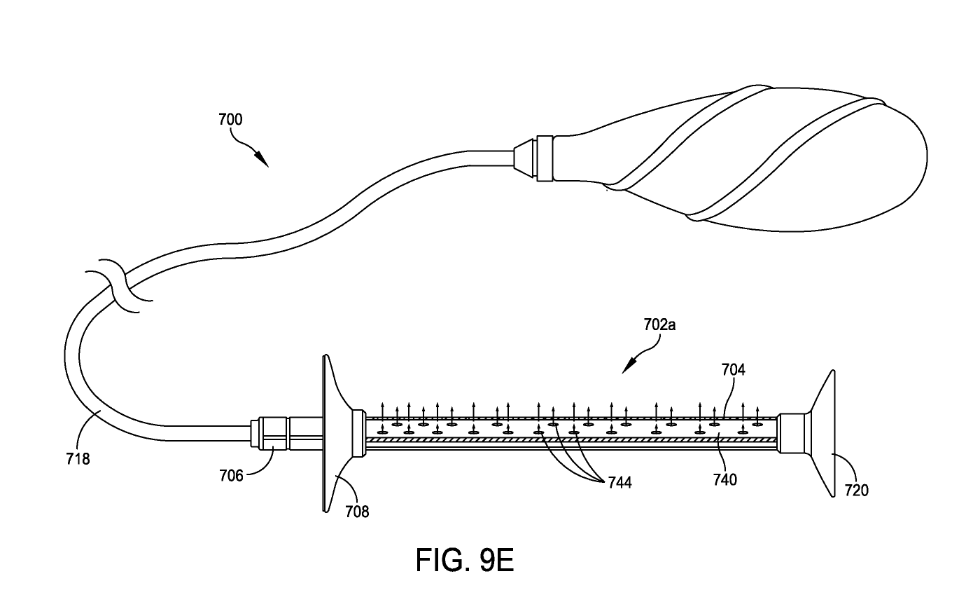

FIG. 9A illustrates one embodiment of a vaginal laxity/uterine prolapse device 702 including a shaft 704 having one or more suction ports 710. The vaginal laxity/uterine prolapse device 702 is similar to the vaginal laxity/uterine prolapse devices described above. The one or more suction ports 710 are disposed along the length of the shaft 704. The suction ports 710 may be evenly spaced and aligned along the shaft 704, unevenly spaced, and/or placed at various rotations and spacing along the shaft 704. The suction ports 710 may comprise any suitable shape, such as, for example, an oblong shape. Although the suction ports 710 are illustrated as oblong suction ports, it will be recognized that the suction ports 710 may comprise any suitable shape. The suction ports 710 are in fluid communication with an internal channel (not shown) defined by the shaft 704. The internal channel is coupled to a suction device, such as, for example, the hand pump 716 illustrated in FIG. 9D. The hand pump 716 is coupled to the proximal end of the shaft 704 by a suction tube 718. A sealing cup 708 is disposed at a proximal end of the shaft 704 and a protective cup 720 is located at a distal end of the shaft 704.

In some embodiments, the shaft 704 is sized and configured to receive a suction distribution cap 740 thereon. One embodiment of a suction distribution cap 740 is illustrated in FIGS. 9B & 9C. The suction distribution cap 740 comprises a hollow elongate tube 742 extending along a longitudinal axis. A plurality of suction holes 744 are disposed along the length of the hollow elongate tube 742. The hollow elongate tube 742 is sized and configured to be placed over the shaft 704 of the vaginal laxity/uterine prolapse device 702. The suction distribution cap 740 distributes suction generated by the suction ports 710 along the length of the suction distribution cap 740. The suction distribution cap 740 is configured to be separated from the shaft 704 for easy cleaning and/or disposable of the suction distribution cap 740. In some embodiments, the suction distribution cap 740 comprises a slit or opening formed along the length of the hollow elongate tube 742 to allow the suction distribution cap 740 to be placed over the shaft 704. In other embodiments, the suction distribution cap 470 is slideably engaged with the shaft 704.

FIG. 9E illustrates one embodiment of a vaginal laxity/uterine prolapse system 700 including the vaginal laxity/uterine prolapse device 702 having the suction distribution cap 740 placed over the shaft 704 and coupled to the hand pump 716. As shown in FIG. 9E, the suction distribution cap 740 distributes the suction generated by the suction ports 710 along the length of the suction distribution cap 740. In some embodiments, the suction distribution cap 740 snaps onto the shaft 704. In other embodiments, the suction distribution cap 740 slides over the shaft 704 (for example, the internal protective cup 720 may be removable to allow the suction distribution cap 740 to slide over the shaft 704 and replaced to lock the suction distribution cap 740 in place). Suction may be generated by, for example, a user by actuating the hand pump 716 to generate air flow through the suction distribution cap 740. The suction distribution cap 740 may be removed after use and cleaned and/or discarded.

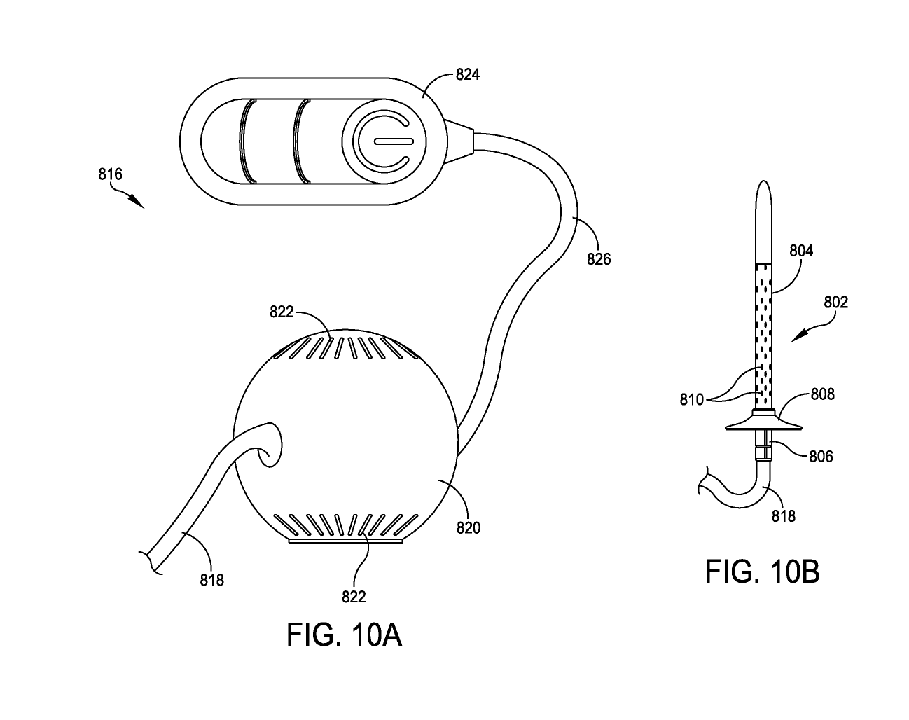

FIG. 10A illustrates one embodiment of an electric pump system 816 configured to generate suction in a vaginal laxity/uterine prolapse device. The electric pump system 816 comprises an electric pump sphere 820 configured to generate negative pressure in a hose 818. The hose 818 is configured to be coupled to a vaginal laxity/uterine prolapse device. The electric pump sphere 820 includes a housing having a plurality of vent holes 822 formed therein. The plurality of vent holes 822 allow air flow out of the electric pump sphere 820 during generation of the negative pressure in the hose 818. The electric pump sphere 820 is coupled to a controller 824 by a control cord 826. The controller 824 is configured to control one or more functions of the electric pump sphere 820, such as, for example, on/off state, suction level, and/or air flow direction.

FIG. 10B illustrates one embodiment of a vaginal laxity/uterine prolapse device 802 configured to couple to the electric pump sphere 820 of FIG. 10A. The vaginal laxity/uterine prolapse device 802 is similar to the vaginal laxity/uterine prolapse devices described above. The vaginal laxity/uterine prolapse device 802 is coupled to the electric pump sphere 820 by the suction hose 818. The electric pump sphere 820 generates negative pressure through the plurality of suction channels 810 formed on the shaft 804 of the vaginal laxity/uterine prolapse device 802.

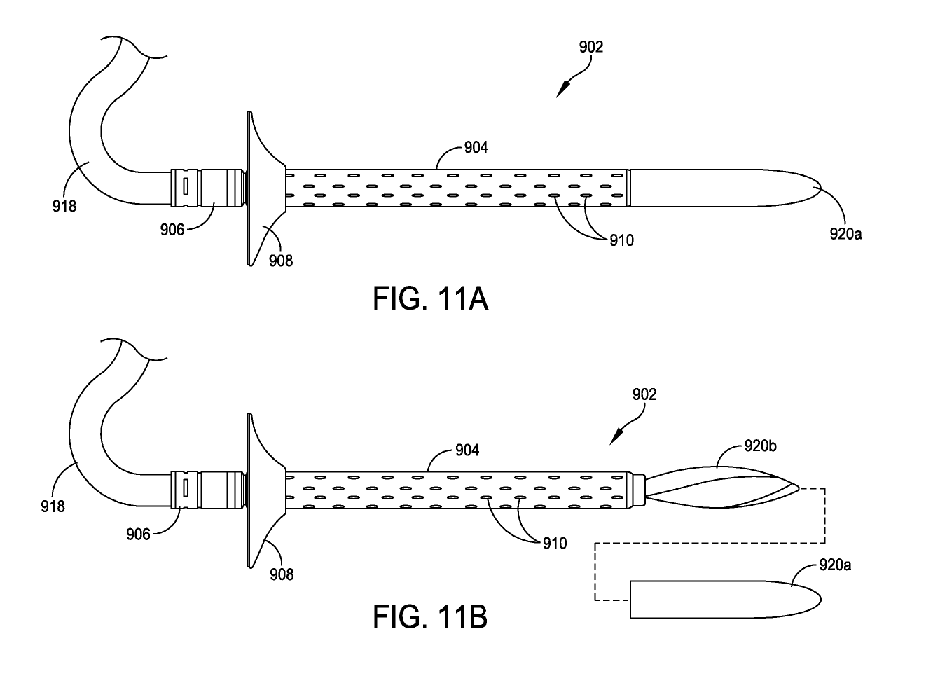

FIG. 11A illustrates one embodiment of a vaginal laxity/uterine prolapse device 902 including a protective blossom cap 920a. The vaginal laxity/uterine prolapse device 902 is similar to the vaginal laxity/uterine prolapse devices described above. A protective blossom cap 920a is coupled to the distal end of the shaft 904. The protective blossom cap 920a is slideably removable from the vaginal laxity/uterine prolapse device to allow deployment of a protective blossom 920b, as shown in FIG. 11B. The protective blossom cap 920a is removed prior to insertion of the vaginal laxity/uterine prolapse device 902 into the vaginal opening. In some embodiments, the protective blossom 920b is configured to expand when the distal portion of the protective blossom 920b is driven into contact with the uterine opening/vaginal wall. The protective blossom 920b serves a similar function to the internal protection cup described above, and protects the uterus and cervix from the negative pressure generated in the vagina. In some embodiments, the protective blossom 920b is expanded by the suction generated in the internal channel (not shown) defined by the shaft 904. The protective blossom 920b may be partially and/or completely expanded by the application of negative pressure to the vaginal laxity/uterine prolapse device 902. In other embodiments, the protective blossom is deployed by a spreading stem.

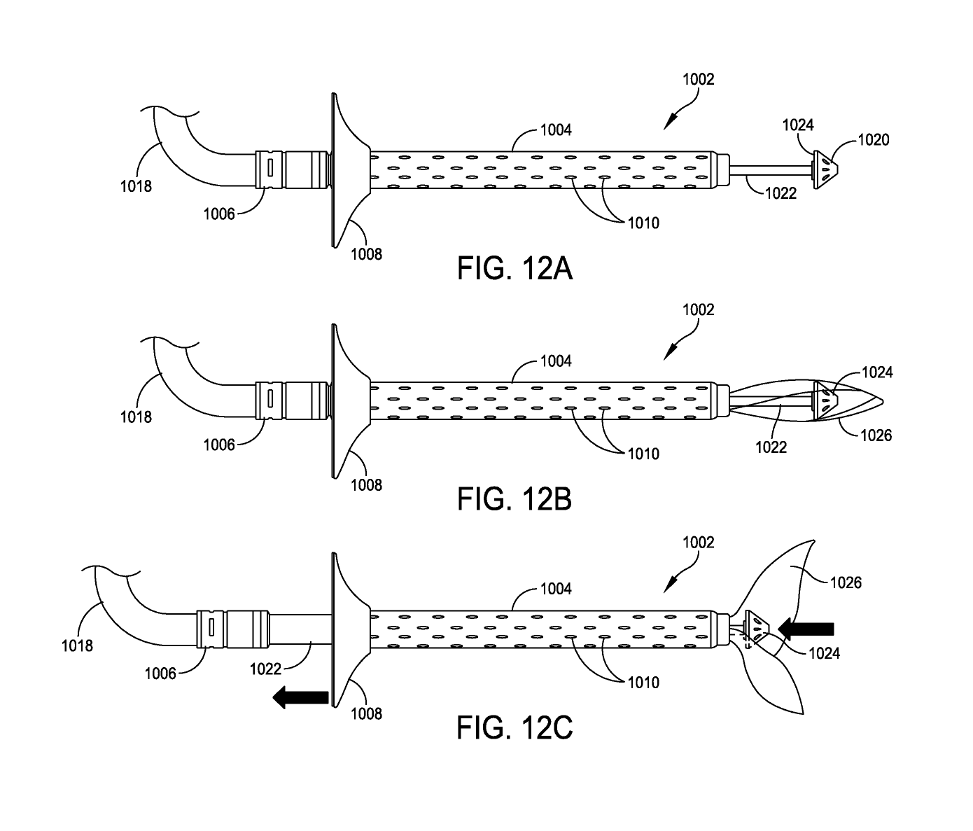

FIG. 12A illustrates one embodiment of a vaginal laxity/uterine prolapse device 1002 including a protective blossom spreading stem 1020. The vaginal laxity/uterine prolapse device 1002 includes a protective blossom spreading stem 1020 (spreading stem) extending distally from the distal end of the shaft 1004. The spreading stem 1020 comprises a stem shaft 1022 extending along a longitudinal axis from the shaft 1004. A spreading cap 1024 is located at a distal end of the stem shaft 1022. In some embodiments, the stem shaft 1022 is concentric with the shaft 1004.

As shown in FIG. 12B, a protective blossom 1026 is disposed over top of the spreading stem 1020. The spreading stem 1020 is actuated from a distal position (shown in FIG. 12B) to a proximal position (shown in FIG. 12C) to deploy the protective blossom 1026. The spreading stem 1020 may be actuated by movement of the handle 1006. For example, in the illustrated embodiment, the stem shaft 1024 extends proximally through shaft 1004 and is coupled to the handle 1006. The handle 1006 is actuated in a proximal direction, resulting in proximal movement of the spreading cap 1024 towards the shaft 1004. As the spreading cap 1024 moves proximally, the protective blossom 1026 expands to a deployed position to protect the uterus and/or the cervix from negative suction generated in the vagina. In some embodiments, the spreading cap 1024 is sized and configured to act as an internal protective cap placed over the cervix after deployment of the uterine protective blossom 1026. In some embodiments, the handle 1006 and the stem shaft 1022 may be moved distally to cause the protective blossom 1026 to transition from the deployed position back to the initial position. In other embodiments, once the protective blossom 1026 is deployed 1026, the spreading stem 1020 is disconnected from the protective blossom 1026.

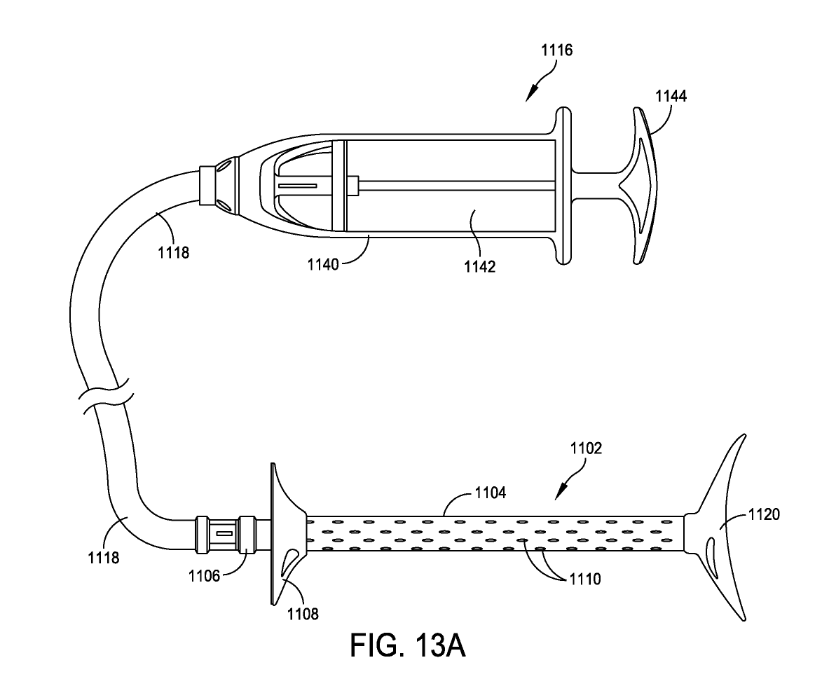

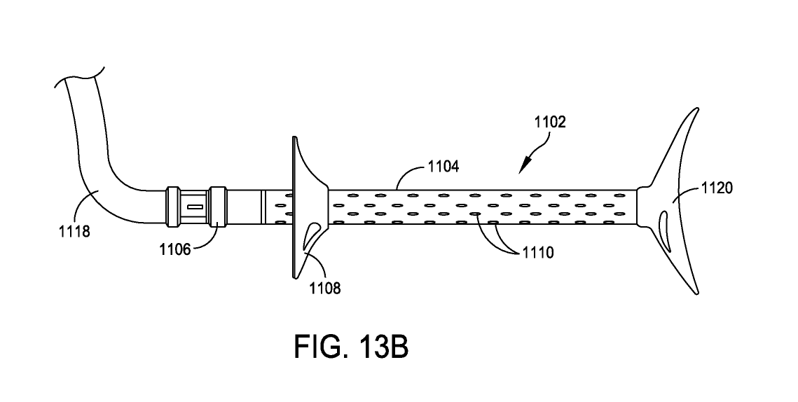

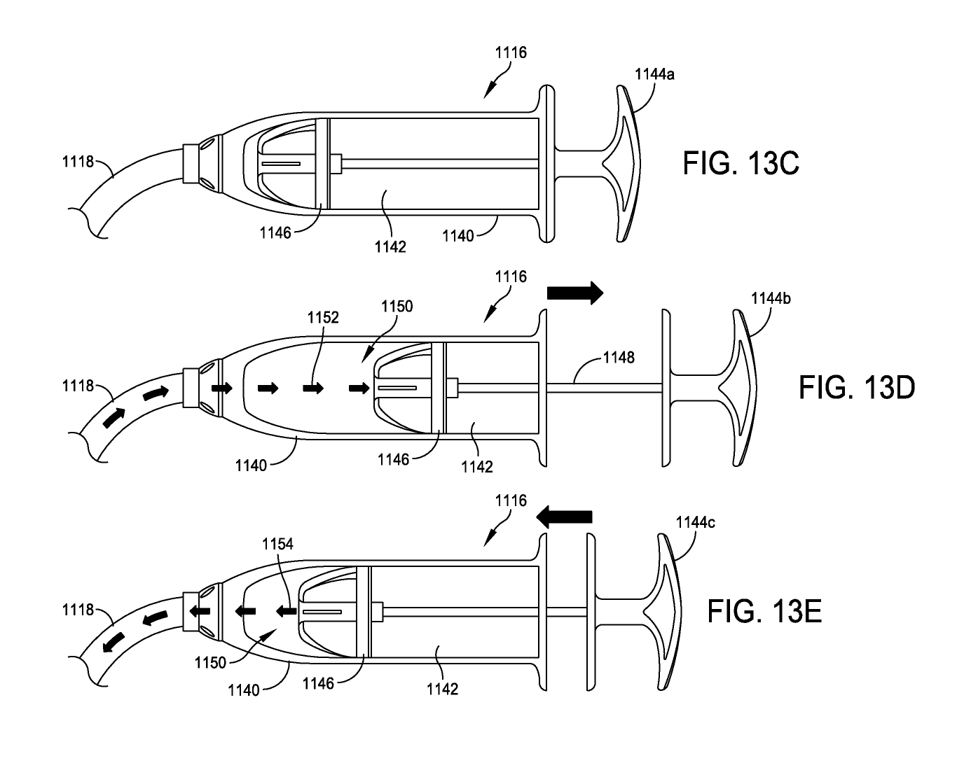

FIGS. 13A-13E illustrate one embodiment of a vaginal laxity/uterine prolapse device 1102 coupled to a manual hand pump 1116. The vaginal laxity/uterine prolapse device 1102 is similar to the vaginal laxity/uterine prolapse devices described above. The vaginal laxity/uterine prolapse device 1102 is coupled to the manual hand pump 1116 by a suction cord 1118. The manual hand pump 1116 is shown with a side wall removed. In operation, a user actuates the handle 1144 of the manual hand pump 1116 to generate negative pressure through the plurality of suction openings 1140 formed in the shaft 1104 of the vaginal laxity/uterine prolapse device 1102. FIGS. 13C-13E illustrate operation of the hand pump 1116. FIG. 13C illustrates the hand pump 1116 in an initial position. The vaginal laxity/uterine prolapse device 1102 is inserted into the vagina with the hand pump 1116 in the initial position. When the shaft 1104 of the vaginal laxity/uterine prolapse device 1102 is inserted, the vagina is sealed by the sealing cup 1108 and the internal protective cup 1120. A user and/or a clinician actuates the hand pump 1116 to generate negative pressure within the vagina. The handle 1144 of the hand pump 1116 is drawn proximally from an initial position 1144a (shown in FIG. 13C) to a proximal-most position 1144B (shown in FIG. 13D). The handle 1144 is coupled to a seal 1146 located within the cavity 1142 by a shaft 1148.

Proximal movement of the seal 1146 (as a result of proximal movement of the handle 1144) causes the seal 1146 to move within the cavity 1142, generating a vacuum space 1150 within the body 1140. The vacuum space 1150 has at least a partial vacuum that causes air 1152 to flow from within the vagina, through the plurality of suction holes 1110, the internal channel, the suction tube 1118 and into the vacuum space 1150, generating a negative pressure within the vagina. The negative pressure generated within the vagina is proportional to the size of the vacuum space 1150 available within the body 1142 of the hand pump 1116. After reaching a proximal-most position (user selected and/or mechanically limited), the handle 1144 of the hand pump 1116 may be driven distally to reverse the flow of air 1154 into the vaginal cavity and reduce the negative pressure generated therein. FIG. 13E illustrates the handle 1144c in a partially distal position corresponding to the release of negative pressure within the vaginal cavity. The hand pump 1116 cycle may be repeated to generate and release negative pressure within the vaginal cavity any number of times, such as, for example, until a specific level of blood flow, a specific time period, and/or until any other threshold parameter is met. In some embodiments, one or more vent openings are formed in the housing of the hand pump 1116 to allow air to be expelled from and/or enter into the cavity 1142 during actuation of the handle 1144.

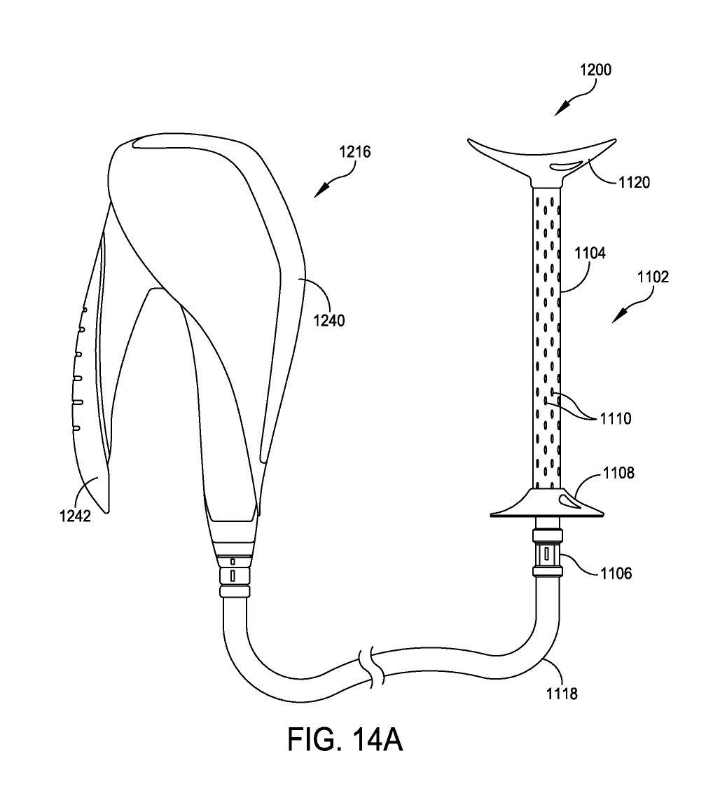

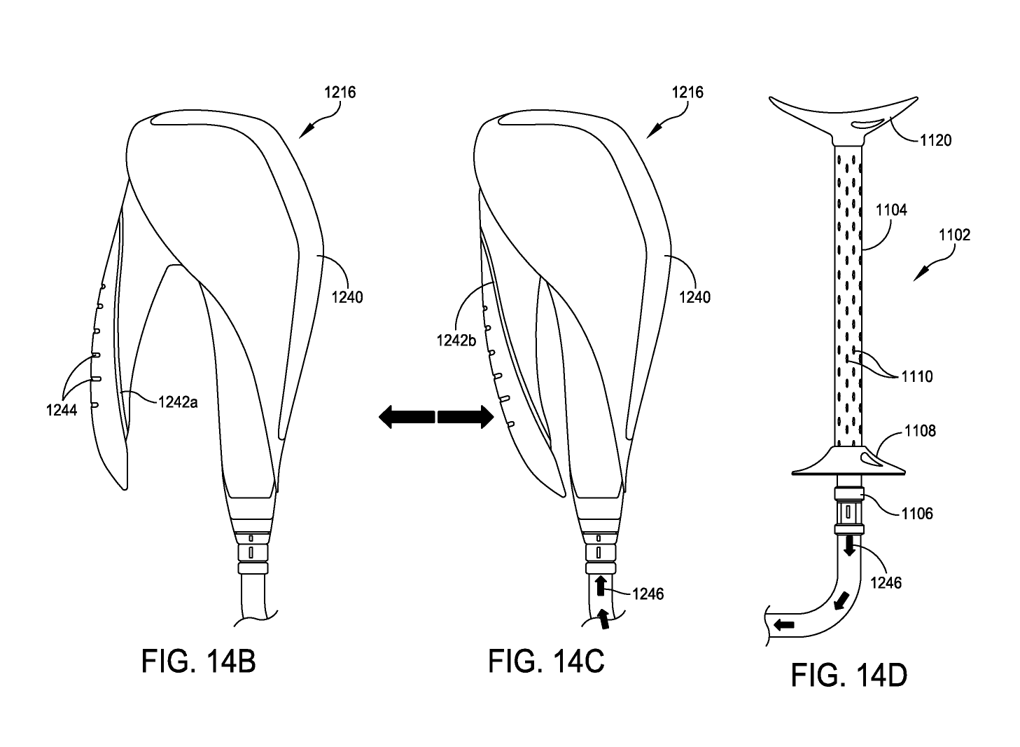

FIG. 14A illustrates one embodiment of a vaginal laxity/uterine prolapse system 1200 including the vaginal laxity/uterine prolapse device 1102 coupled to a hand suction pump 1216. The vaginal laxity/uterine prolapse device 1102 is coupled to the hand suction pump 1216 by a suction hose 1118. The hand suction pump 1216 is configured to be operated with one hand. The hand pump 1216 comprises a handle 1240 having a lever 1242 pivotally coupled thereto. The lever 1242 is actuateable from a first position (shown in FIG. 14B) to a second position (shown in FIG. 14C). In some embodiments, the lever 1242 comprise a plurality of gripping features 1244 formed thereon.

FIGS. 14B and 14C illustrate operation of the hand pump 1216. The lever 1242 of the hand pump 1216 is illustrated in an initial position 1242a in FIG. 14B. The lever 1242a is in the initial position during insertion of the vaginal laxity/uterine prolapse device 1102 into the vaginal opening. After insertion of the shaft 1104 into the vagina, the lever 1242 is actuated from the first position 1242a to the second position 1242b. Actuation of the lever 1242 towards the handle 1240 generates negative pressure within the vagina through air flow 1246 out of the vaginal laxity/uterine prolapse device 1102. For example, in some embodiments, the hand pump 1216 shares a similar internal construction to the hand pump 1116. Releasing and/or actuating the lever 1242 away from the handle 1240 releases the negative pressure within the vagina. In other embodiments, repeated actuation of the lever 1242 increases the negative pressure within the vagina. FIG. 14D illustrates the vaginal laxity/uterine prolapse device 1102 applying suction caused by the flow of air 1246.

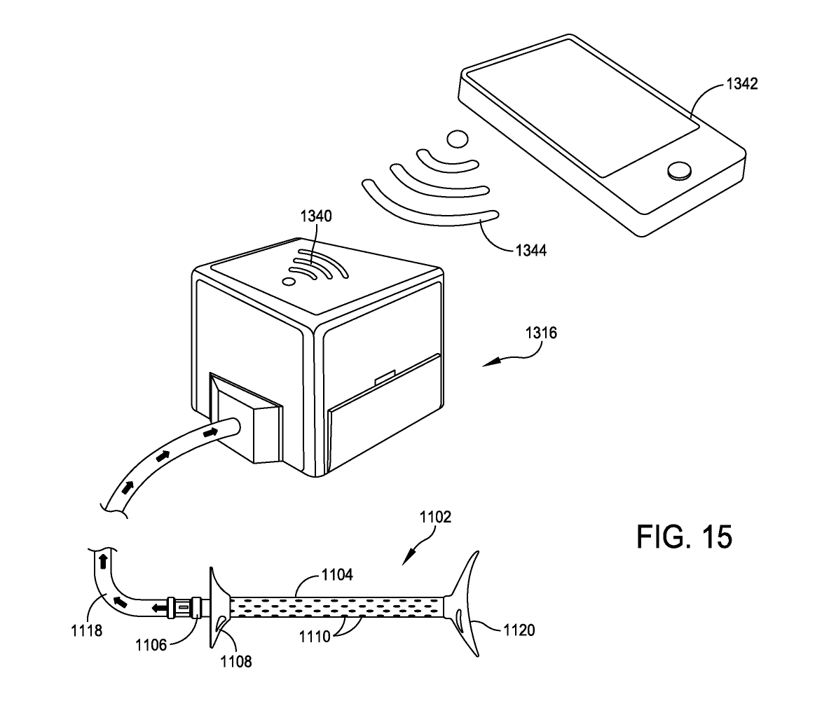

FIG. 15 illustrates one embodiment of the vaginal laxity/uterine prolapse device 1102 coupled to a wirelessly controlled electric pump 1316. The vaginal laxity/uterine prolapse device 1102 is coupled to the electric pump 1316 by a suction tube 1118. The electric pump 1316 comprises one more systems configured to generate a negative pressure through the suction tube 1118 and, by extension, through the plurality of suction channels 1110 formed on the shaft 1104. The electric pump 1316 further comprises one or more control circuits for controlling the negative pressure generated, such as, for example, control circuits to control the on/off state of the pump, the pressure level of the negative pressure generated, and/or the direction of air flow. In some embodiments, one or more of the control circuits are coupled to a wireless receiver 1340. The wireless receiver 1340 is configured to receive wireless control signals 1344, for example, from a wireless controller 1342. The wireless controller 1342 is configured to control one or more of the functions of the electric pump, such as, for example, the on/off state of the pump, the pressure level of the negative pressure generated, and/or the direction of air flow. In some embodiments, the controller 1342 receives signals from the electric pump 1316 and displays information to a user via a screen. For example, the controller 1342 may be configured to display a time of operation, a suction strength indicator, a battery power indication, and/or any other suitable information.

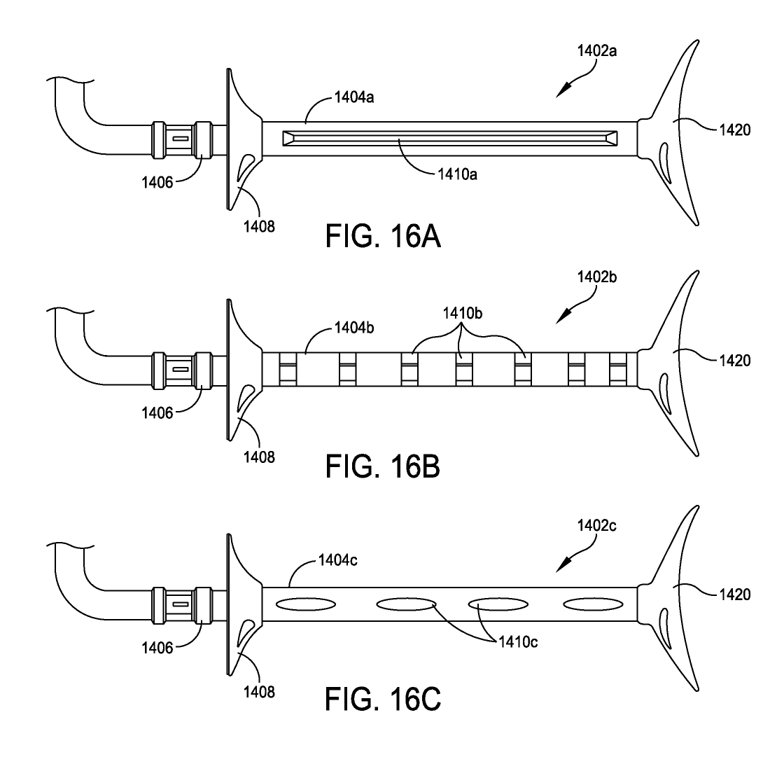

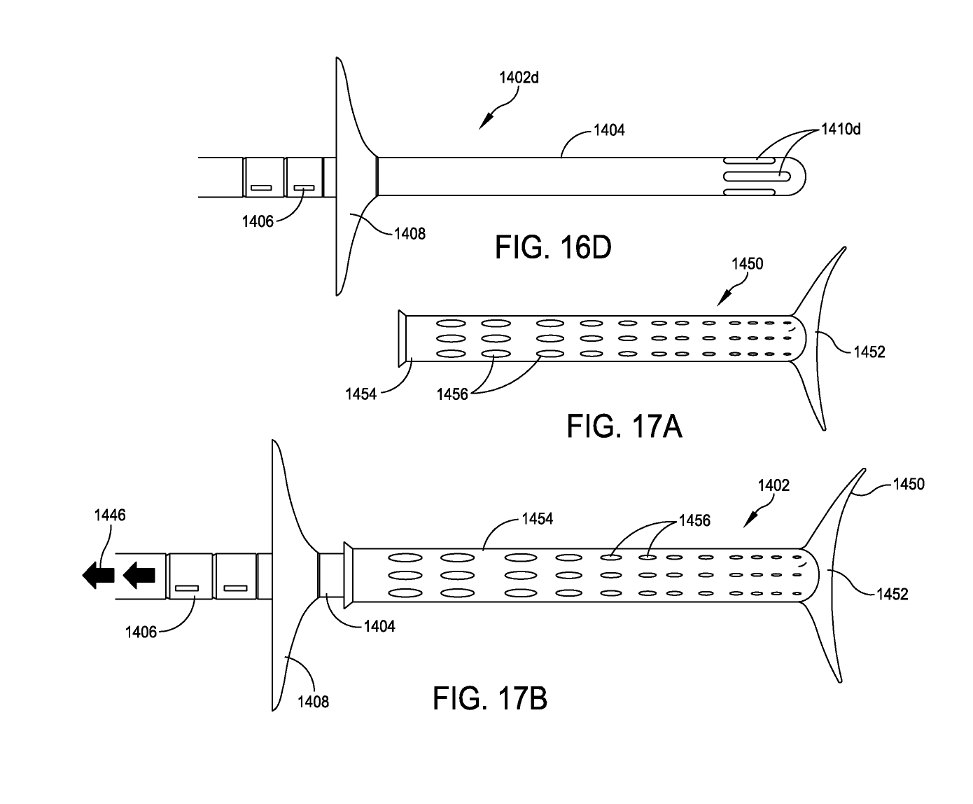

FIGS. 16A-16D illustrate embodiments of vaginal laxity/uterine prolapse devices having various suction port patterns. FIG. 16A illustrates one embodiment of a vaginal laxity/uterine prolapse device 1402a having a shaft 1404a extending along a longitudinal axis. The shaft is coupled to a handle 1406 and has a sealing cup 1408 disposed between the shaft 1404a and the handle 1406. An internal protective cup 1420 is coupled to the distal end of the shaft 1404a. The shaft 1404 defines an elongate suction channel 1410a formed thereon. The elongate suction channel 1410a comprise a channel, or trough, formed in the shaft 1404 and extends through a surface of the shaft 1404 to allow fluid communication between the outer surface of the shaft 1404 and an internal channel (not shown). FIG. 16B illustrates one embodiment of the vaginal laxity/uterine prolapse device 1402b having a shaft 1404b including a plurality of concentric suction rings 1410b formed along the length of the shaft 1404b. Each of the suction rings 1410b includes at least one suction channel 1411 formed through the surface of the shaft 1404b. FIG. 16C illustrates one embodiment of the vaginal laxity/uterine prolapse device 1402c having a shaft 1404c including a plurality of elongate suction holes 1410c. The plurality of elongate suction holes 1410c are formed through the outer surface of the shaft 1404c. FIG. 16D illustrates one embodiment of the vaginal laxity/uterine prolapse device 1402d having a shaft 1404d including a plurality of suction channel 1410d formed at a distal end of the shaft 1404d.

FIG. 17A illustrates one embodiment of a suction distribution cover 1450 configured to couple to the shaft 1404 of a vaginal laxity/uterine prolapse device 1402. The suction distribution cover 1450 comprises a plurality of suction distribution holes 1456 formed along the shaft 1454 of the suction distribution cover 1450. The shaft 1454 defines a hollow shaft having an opening formed at a proximal end. The opening is sized and configured to fit over the shaft 1404 of the vaginal laxity/uterine prolapse device 1402 (see FIG. 17B). The suction distribution cover 1450 includes an internal protective cup 1452. In some embodiments, the plurality of suction distribution holes 1456 have graduated perforation sizes to evenly distribute suction away from one or more suction ports. For example, in the illustrated embodiment, the suction distribution cover 1450 includes a plurality of suction distribution holes 1456 having graduated perforation sizes, with the smallest perforations being located at a distal end of the suction distribution cover 1450 and the largest perforation being located at a proximal end of the suction distribution cover 1450 to distribute suction generated by, for example, the vaginal laxity/uterine prolapse device 1402d illustrated in FIG. 16D. In some embodiments, the suction distribution cover 1450 comprises a disposable cover, such as, for example, a rubber condom-like cover configured to be placed over the shaft 1404 of the vaginal laxity/uterine prolapse device 1402.

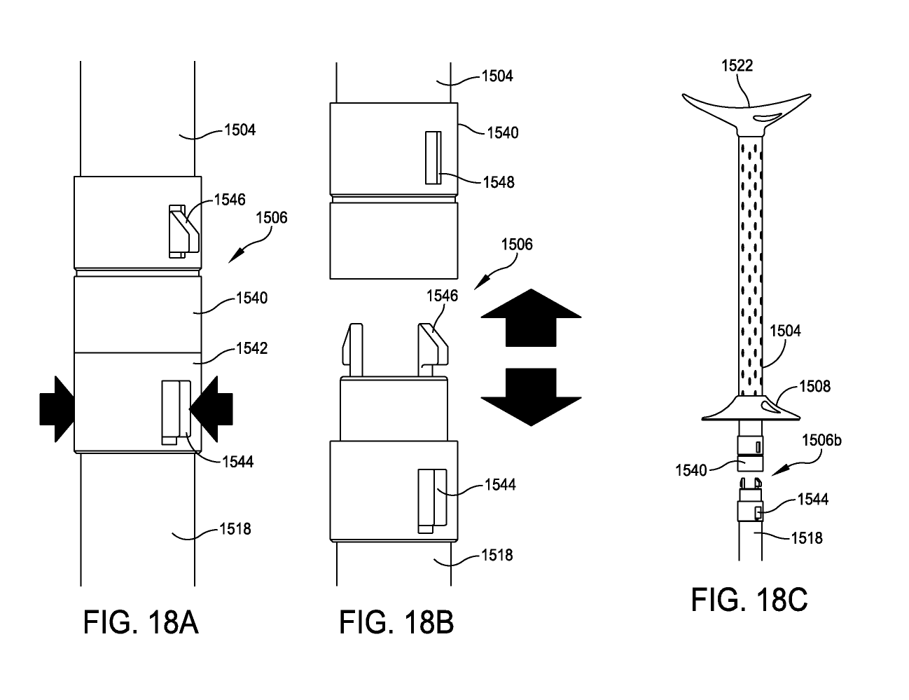

FIGS. 18A-18C illustrate one embodiment of a quick connect system 1506 for coupling a vaginal laxity/uterine prolapse device 1502 to a suction hose 1518. The quick connect system 1506a comprises a first connector 1540 coupled to the vaginal laxity/uterine prolapse device 1502, for example, at a proximal end of the shaft 1504. The first connector 1540 is configured to couple to a second connector 1542 coupled to the suction hose 1518. The first connector 1540 comprises one or more holes 1548 sized and configured to receive connection projections 1546 therethrough. The connection projections 1546 are coupled to a button 1544 formed on the second connector 1542. Depressing the button 1544 causes the connection projections 1546 to move inwards towards a central axis and allows the projections 1546 to be released from the holes 1548 (as shown in FIG. 18B). When the first connector 1540 is slideably interfaced with the second connector 1542, the projections 1546 are depressed until they are aligned with the holes 1548. When the projections 1546 are aligned with the holes 1548, the projections resume their un-depressed state through the holes 1548, locking the first connector 1540 to the second connector 1542. FIG. 18C illustrates one embodiment of vaginal laxity/uterine prolapse device 1502 having a first connector 1540 of the quick connect system 1506 coupled to a distal end of the shaft 1504. The quick connect system 1506 allows the vaginal laxity/uterine prolapse device 1502 to be easily removed from the suction hose 1508 for cleaning, maintenance, and/or storage.

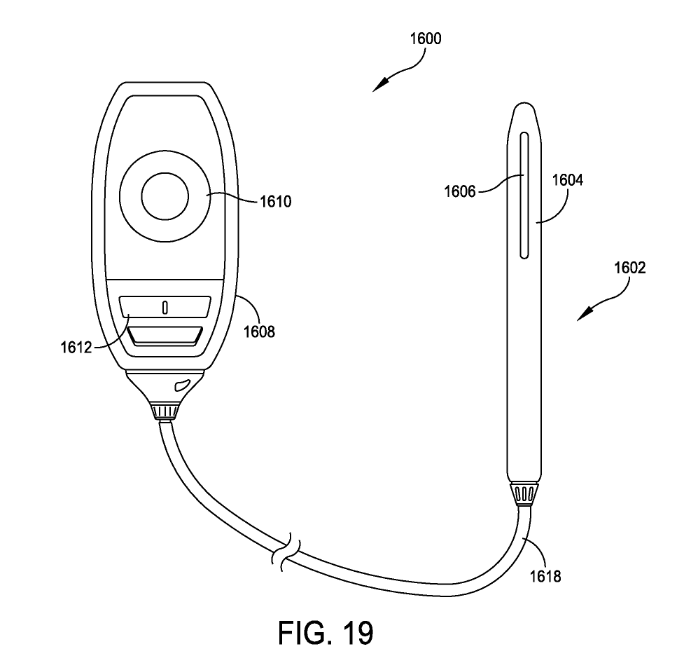

FIG. 19 illustrates one embodiment of a pressure measuring device 1600 sized and configured for insertion into a vagina. The pressure measuring device 1600 is configured to be used in conjunction with one or more of the vaginal laxity/uterine prolapse devices described in conjunction with FIGS. 1-18C. The pressure measuring device 1600 comprises a shaft 1604 sized and configured for insertion into a vagina. The shaft includes at least one sensor 1606 coupled thereto. The at least one sensor 1606 may be coupled to any portion of the shaft 1604, such as, for example, a distal portion, a proximal portion, or a middle portion of the shaft 1604. The shaft 1604 and the at least one sensor 1606 are coupled to a controller 1608 by a signal cable 1618. The controller 1608 receives a signal from the at least one sensor 1606 and displays a pressure reading on a screen 1610. The controller 1608 further includes a power switch 1612.

In some embodiments, the pressure measuring device 1600 is used in conjunction with one of the vaginal laxity/uterine prolapse devices described above. The pressure measure device 1600 may be inserted simultaneously with the vaginal laxity/uterine prolapse device to measure pressure generated within the vagina by the vaginal laxity/uterine prolapse device. In other embodiments, the pressure measuring device 1600 is used separately from the vaginal laxity/uterine prolapse device to measure short term and/or long term increase in vaginal blood flow, muscle tightness, and/or other factors of the vagina. For example, in one embodiment, the pressure measuring device 1600 is inserted through the vaginal opening immediately following a treatment session utilizing a vaginal laxity/uterine prolapse device. The pressure measure device 1600 provides a pressure reading indicative of the increased vaginal blood flow developed by the vaginal laxity/uterine prolapse device. The pressure measuring device 1600 can be used to indicate whether a desired level of blood flow and/or muscle tightness has been achieved.

In some embodiments, the pressure measuring device 1600 and/or a vaginal laxity/uterine prolapse device includes a wireless communication module (not shown). The wireless communication module is configured to communicate with a computing device, such as, for example, a smartphone. The wireless communication module provides a signal indicative of the pressure achieved at a given internal diameter of the vagina. The computing device is configured to provide a record of pressure achieved at a given internal diameter, e.g., “tightness,” to a user. In some embodiments, tightness is proportional to the positive pressure required to achieve a given vaginal internal diameter, for example, vaginal tone (“tightness”) pressure/(internal diameter). In some embodiments, the wireless communication module is configured to receive inputs from the computing device, such as, for example, suction control signals. The suction control signals may be received in response to user input and/or pre-programmed response algorithms.

In other embodiments, the pressure measuring device 1600 is used to determine the long term effects of the vaginal laxity/uterine prolapse device. For example, the pressure measuring device 1600 may be used in between treatment sessions using a vaginal laxity/uterine prolapse device to measure the gradual increase of pressure within the vagina caused by increased muscle size and hypertrophy of the vaginal/pelvic region. The pressure measuring device 1600 may be used to measure progress, adjust a treatment regimen, and/or to otherwise monitor the use of the vaginal laxity/uterine prolapse device over an extended period.

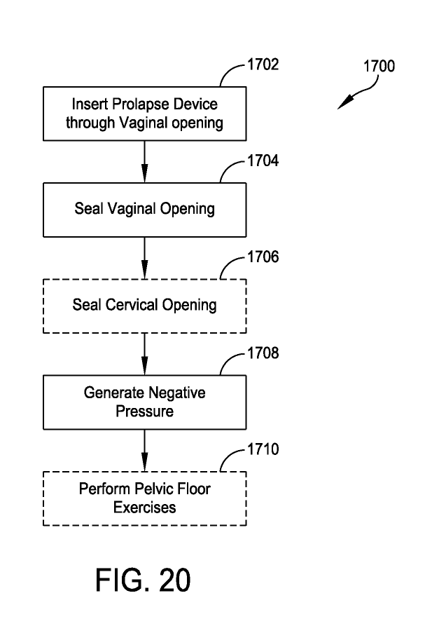

FIG. 20 is a flowchart illustrating one embodiment of a method 1700 for generating edema and/or hypertrophy of a vaginal wall. The method and associated devices provides both immediate (e.g., within minutes) and chronic (e.g., over days to weeks of repetitive application) decrease in the compliance of the vagina, increasing the frictional resistance to penile penetration (i.e., “tightness”) to treat vaginal laxity and conversely increasing the frictional resistance to retrograde displacement of the uterus to decrease the frequency and severity of uterine prolapse. The method 1700 includes a first step 1702 of inserting a vaginal laxity/uterine prolapse device through a vaginal opening of a patient/user. The vaginal laxity/uterine prolapse device includes a shaft sized and configured for insertion into the vagina through the vaginal opening. The shaft has a plurality of suction openings formed through the shaft and coupled to an internal channel defined by the shaft. The vaginal laxity/uterine prolapse device may be, for example, any one of the vaginal laxity/uterine prolapse devices described in conjunction with FIGS. 1-18C.

In a second step 1704 of the method 1700, the vagina is sealed. The vagina may be sealed by, for example, a sealing cup located at a proximal end of the shaft and/or a sealing profile defined by the proximal end of the shaft. The sealing cup and/or the sealing profile are sized and configured to seal the vaginal opening. In an optional third step 1706, a cervix is sealed by an internal protective cup located at a distal end of the shaft. The internal protective cup is sized and configured to fit at least partially over the cervix and to protect the cervix and/or uterus from negative pressure generated within the vagina.

In a fourth step 1708, negative pressure is generated inside of the vagina to cause edema and/or hypertrophy of the vaginal walls. The negative pressure is generated by a suction device located externally to the vagina. In some embodiments, the suction device is coupled to the shaft of the vaginal laxity/uterine prolapse device by a suction tube. The suction device generates negative pressure through the plurality of suction openings formed on the shaft. The negative pressure causes increased blood flow to the vaginal walls and edema and/or hypertrophy of the vaginal walls.

In an optional fifth step 1710, a user performs one or more pelvic floor exercises simultaneously and/or alternatively with the generation of negative pressure. For example, in some embodiments, a user may perform pelvic floor exercises such as kegel exercises simultaneously with the generation of suction by the vaginal laxity/uterine prolapse device. In other embodiments, the user may alternate the suction and the pelvic floor exercises.

In various embodiments, a system is disclosed. The system comprises a prolapse device. The prolapse device includes a shaft sized and configured for insertion into a vagina. The shaft extends longitudinally from a proximal end to a distal end. The shaft defines an internal channel. At least one suction hole is formed through the shaft to provide fluid communication between an outer surface of the shaft and the internal channel. A handle is coupled to the proximal end of the shaft. A sealing cup is located between the handle and the shaft. The sealing cup is sized and configured to fit over and seal a vaginal opening. The system further comprises a suction device coupled to the internal channel defined by the shaft and configured to generate suction through the plurality of holes.

In some embodiments, the prolapse device comprises an internal protective cup coupled to a distal end of the shaft. The internal protective cup is sized and configured to contact an internal opening within the body cavity. The internal protective cup comprises a deformable shape-memory material. The internal protective cup is sized and configured to seal a cervical opening. In some embodiments, the distal end of the shaft comprises a blossom spreading stem shaft and the internal protective cup comprises a protective blossom.

In some embodiments, the proximal end of the shaft defines an internal seating profile. The internal seating profile comprises a diameter sufficient to seal a vaginal opening. In some embodiments, the suction device comprises a hand-operated pump. In other embodiments, the suction device comprises an electric pump.

In some embodiments, the system further comprises a suction distribution cover sized and configured to cover the shaft of the prolapse device. The suction distribution cover extends longitudinally. The shaft has a plurality of holes formed therethrough. The suction distribution cover comprises a hollow cylinder having an internal diameter greater than or equal to the diameter of the shaft. In some embodiments, the system includes at least one electrical contact formed on the shaft. The at least one electrical contact may comprise a bipolar electrical contact or a monopolar electrical contact. In some embodiments, the shaft has an adjustable length.

In various embodiments, a vaginal laxity/uterine prolapse device is disclosed. The vaginal laxity/uterine prolapse device comprises a shaft sized and configured for insertion into a body cavity, a handle coupled to the proximal end of the shaft, and a sealing cup located between the handle and the shaft. The shaft extends longitudinally from a proximal end to a distal end. The shaft defines an internal channel. At least one suction hole is formed through the shaft to provide fluid communication between an outer surface of the shaft and the internal channel. The sealing cup is sized and configured to fit over and seal an external opening of the body cavity.

In some embodiments, the vaginal laxity/uterine prolapse device comprises an internal protective cup coupled to a distal end of the shaft. The internal protective cup is sized and configured to contact an internal opening within the body cavity. In some embodiments a suction distribution cover is disposed over at least a portion of the shaft. The suction distribution cover comprises a plurality of openings sized and configured to distribute suction generated by the at least one suction hole over a length of the suction distribution cover.

In various embodiments, a method is disclosed. The method comprises inserting a shaft into a vagina. The shaft extends longitudinally from a proximal end to a distal end and defines an internal channel. At least one suction hole is formed through the shaft to provide fluid communication between an outer surface of the shaft and the internal channel. The method further comprises sealing the vaginal opening with a sealing cup located at a proximal end of the shaft. The sealing cup sized and configured to fit over and seal a vaginal opening. The method further comprises generating negative pressure within the vagina through the at least one suction hole. In some embodiments, the method includes performing one or more pelvic floor exercises simultaneously with the generation of negative pressure within the vagina

Although the subject matter has been described in terms of exemplary embodiments, it is not limited thereto. Rather, the appended claims should be construed broadly, to include other variants and embodiments, which may be made by those skilled in the art.

What is claimed is:

1. A system, comprising:
a device comprising:
a shaft sized and configured for insertion into a vagina, the shaft extending longitudinally from a proximal end to a distal end, wherein the shaft defines an internal channel, and wherein at least one suction hole is formed through the shaft to provide fluid communication between an outer surface of the shaft and the internal channel;
a handle coupled to the proximal end of the shaft; and
a sealing cup located between the handle and the shaft, the sealing cup sized and configured to fit over and seal a vaginal opening; and
a suction device coupled to the internal channel defined by the shaft and configured to generate suction through the plurality of holes.

2. The system of claim 1, wherein the device comprises an internal protective cup coupled to a distal end of the shaft, the internal protective cup sized and configured to contact an internal opening within the body cavity.

3. The system of claim 2, wherein the internal protective cup comprises a deformable shape-memory material.

4. The system of claim 2, wherein the internal protective cup is sized and configured to seal a cervical opening.

5. The system of claim 2, wherein the distal end of the shaft comprises a blossom spreading stem shaft and the internal protective cup comprises a protective blossom.

6. The system of claim 1, wherein the proximal end of the shaft defines an internal seating profile.

7. The system of claim 6, wherein the internal seating profile comprises a diameter sufficient to seal a vaginal opening.

8. The system of claim 1, wherein the suction device comprises a hand-operated pump.

9. The system of claim 1, wherein the suction device comprises an electrically-powered pump.

10. The system of claim 1, further comprising a suction distribution cover sized and configured to cover the shaft of the device, the suction distribution cover extending longitudinally and having a plurality of holes formed therethrough.

11. The system of claim 10, wherein the suction distribution cover comprises a hollow cylinder having an internal diameter greater than or equal to the diameter of the shaft.

12. The system of claim 1, comprising at least one electrical contact formed on the shaft.

13. The system of claim 12, wherein the at least one electrical contact comprises a bipolar electrical contact.

14. The system of claim 12, wherein the at least one electrical contact comprises a monopolar electrical contact.

15. The system of claim 1, wherein the shaft comprises an adjustable length.

16. A device comprising:
a shaft sized and configured for insertion into a body cavity, the shaft extending longitudinally from a proximal end to a distal end, wherein the shaft defines an internal channel, and wherein at least one suction hole is formed through the shaft to provide fluid communication between an outer surface of the shaft and the internal channel;
a handle coupled to the proximal end of the shaft; and
a sealing cup located between the handle and the shaft, the sealing cup sized and configured to fit over and seal an external opening of the body cavity.

17. The device of claim 16, comprising an internal protective cup coupled to a distal end of the shaft, the internal protective cup sized and configured to contact an internal opening within the body cavity.

18. The device of claim 17, comprising a suction distribution cover disposed over at least a portion of the shaft, the suction distribution cover comprising a plurality of openings sized and configured to distribute suction generated by the at least one suction hole over a length of the suction distribution cover.

19. A method comprising:
inserting a shaft into a vagina, the shaft extending longitudinally from a proximal end to a distal end and defining an internal channel, wherein at least one suction hole is formed through the shaft to provide fluid communication between an outer surface of the shaft and the internal channel;
sealing the vaginal opening with a sealing cup located at a proximal end of the shaft, the sealing cup sized and configured to fit over and seal a vaginal opening; and
generating negative pressure within the vagina through the at least one suction hole.

20. The method of claim 19, further comprising performing one or more pelvic floor exercises simultaneously with the generation of negative pressure within the vagina.
